(19) United States
(12) Patent Application Publication
(10) Pub. No.: US 2025/0390141 A1
(43) Pub. Date: Dec. 25, 2025

(54) ELECTRONIC DEVICE HAVING SENSOR AND METHOD FOR OBTAINING INFORMATION THEREOF

(71) Applicant: Samsung Electronics Co., Ltd., Suwon-si (KR)

(72) Inventors: Daehyeong LIM, Suwon-si (KR); Minho PARK, Suwon-si (KR); Joongwoo AHN, Suwon-si (KR); Younghyun KIM, Suwon-si (KR)

(21) Appl. No.: 19/205,280

(22) Filed: May 12, 2025

Related U.S. Application Data
(63) Continuation of application No. PCT/KR2025/003953, filed on Mar. 27, 2025.

(30) Foreign Application Priority Data
Jun. 21, 2024 (KR) 10-2024-0081431
Jul. 9, 2024 (KR) 10-2024-0090571
Sep. 6, 2024 (KR) 10-2024-0121227

(51) Int. Cl. G06F 1/16; G06F 3/01

(52) U.S. Cl. CPC G06F 1/163; G06F 1/1698; G06F 3/015

(57) ABSTRACT

An electronic device is provided. The electronic device includes a housing including a first surface and a second surface opposite the first surface, a third surface substantially surrounding a space between the first surface and the second surface and forming a lateral surface of the wearable device, a display received in the housing to be seen through the first surface, a printed circuit board disposed between the display and the second surface, and at least one biometric sensor disposed on the printed circuit board to face the second surface and including multiple light-emitting elements and multiple light-receiving elements, wherein the multiple light-emitting element include a first light-emitting element disposed in a peripheral area of the printed circuit board and configured to emit first light in a first specified band, and a second light-emitting element disposed in a center area of the printed circuit board and configured to emit second light in a second specified band at least partially different from the first specified band, and a wherein the multiple light-receiving elements include a first light-receiving element, and a second light-receiving element configured to receive light emitted from the first light-emitting element and the second light-emitting element and reflected from a portion of a user's body, the first light-emitting element being disposed in the peripheral area to be adjacent to each of the first light-receiving element and the second light-receiving element.

### CROSS-REFERENCE TO RELATED APPLICATION(S)

This application is a continuation application, claiming priority under § 365 (c), of an International application No. PCT/KR2025/003953, filed on Mar. 27, 2025, which is based on and claims the benefit of a Korean patent application number 10-2024-0081431, filed on Jun. 21, 2024, in the Korean Intellectual Property Office, of a Korean application number 10-2024-0090571, filed on Jul. 9, 2024, in the Korean Intellectual Property Office, and of a Korean application number 10-2024-0121227, filed on Sep. 6, 2024, in the Korean Intellectual Property Office, the disclosure of each of which is incorporated by reference herein in its entirety.

### TECHNICAL FIELD

The disclosure relates to an electronic device including a sensor and a method for obtaining information using same.

### BACKGROUND ART

In accordance with developments of digital technologies, various types of electronic devices, such as a mobile communication terminal, a personal digital assistant (PDA), an electronic notebook, a smartphone, a tablet personal computer (PC), and a wearable device, have been widely used. The electronic devices have been continuously improved in terms of hardware and/or software of the electronic devices to support and expand functions thereof.

Recent wearable electronic devices are equipped with optical sensors, which are utilized to measure various biometric signals such as heart rate, blood oxygen saturation (SpO₂), or advanced glycation end-products, enabling the acquisition of biometric information.

The above information is presented as background information only to assist with an understanding of the disclosure. No determination has been made, and no assertion is made, as to whether any of the above might be applicable as prior art with regard to the disclosure.

### DISCLOSURE OF INVENTION

### Technical Problem

Aspects of the disclosure are to address at least the above-mentioned problems and/or disadvantages and to provide at least the advantages described below. Accordingly, an aspect of the disclosure is to provide an electronic device relates to a biometric signal measurement technology using an optical sensor and provides, a more accurate method of measuring advanced glycation end-products among biometric signals.

Additional aspects will be set forth in part in the description which follows and, in part, will be apparent from the description, or may be learned by practice of the presented embodiments.

### Solution to Problem

In accordance with an aspect of the disclosure, a wearable device is provided. The wearable device includes a housing including a first surface and a second surface opposite the first surface, and a third surface substantially surrounding a space between the first surface and the second surface and forming a lateral surface of the wearable device, a display received in the housing to be seen through the first surface, a printed circuit board disposed between the display and the second surface, at least one biometric sensor disposed on the printed circuit board to face the second surface and including multiple light-emitting elements and multiple light-receiving elements, wherein the multiple light-emitting elements include a first light-emitting element disposed in a peripheral area of the printed circuit board and configured to emit light in a first specified band, and a second light-emitting element disposed in a center area of the printed circuit board and configured to emit second light in a second specified band at least partially different from the first specified band, and wherein the multiple light-receiving elements include a first light-receiving element, and a second light-receiving element configured to receive light emitted from the first light-emitting element and the second light-emitting element and reflected from a portion of a user's body, the first light-emitting element being disposed in the peripheral area to be adjacent to each of the first light-receiving element and the second light-receiving element.

In accordance with another aspect of the disclosure, an electronic device is provided. The electronic device includes a housing having a transparent cover coming in contact with the body of a user of the electronic device, multiple light-emitting elements including a first light-emitting element configured to emit ultraviolet (UV) light through the transparent cover and a second light-emitting element separated from the first light-emitting element by means of a partition wall and configured to emit at least one of visible light or infra-red (IR) light through the transparent cover, multiple light-receiving elements including a first light-receiving element and a second light-receiving element configured to detect unfiltered UV light and filtered UV light, emitted from the multiple light-emitting elements and reflected by a portion of the user's body, respectively, memory storing one or more computer programs, and one or processors communicatively coupled to the memory, wherein the one or more computer programs include computer-executable instructions that, when executed by the one or more processors individually or collectively, cause the electronic device to acquire first biometric information associated with the user based on the unfiltered UV light and the filtered UV light corresponding to the UV light emitted by the first light-emitting element, and acquire second biometric information based on the unfiltered UV light and/or the filtered UV light corresponding to at least one of the visible light or the IR light emitted by the second light-emitting element.

In accordance with another aspect of the disclosure, one or more non-transitory computer-readable storage media storing one or more computer programs including computer-executable instructions that, when executed by one or more processors of an electronic device individually or collectively, cause the electronic device to perform operations are provided. The operations include acquiring first biometric information associated with a user, based on unfiltered UV light and filtered UV light corresponding to UV light emitted by a first light-emitting element of the electronic device, and acquiring second biometric information, based on the unfiltered UV light and/or the filtered UV light corresponding to at least one of visible light or infrared light emitted by a second light-emitting element of the electronic device.

Other aspects, advantages, and salient features of the disclosure will become apparent to those skilled in the art from the following detailed description, which, taken in conjunction with the annexed drawings, discloses various embodiments of the disclosure.

### BRIEF DESCRIPTION OF DRAWINGS

The above and other aspects, features, and advantages of certain embodiments of the disclosure will be more apparent from the following description taken in conjunction with the accompanying drawings, in which:

FIG. 1 is a block diagram illustrating an electronic device in a network environment according to an embodiment of the disclosure;

FIG. 2 is a perspective diagram illustrating a plane or cross section of a wearable electronic device according to an embodiment of the disclosure;

FIG. 3 is a view illustrating an optical sensor of an electronic device according to an embodiment of the disclosure;

FIG. 4 is a view illustrating an optical sensor of an electronic device according to an embodiment of the disclosure;

FIG. 5 is a view illustrating an optical sensor of an electronic device according to an embodiment of the disclosure;

FIG. 6 is a view illustrating an optical sensor of an electronic device according to an embodiment of the disclosure;

FIGS. 7A, 7B, and 8 are views illustrating an operation of an optical sensor of an electronic device according to various embodiments of the disclosure;

FIGS. 9, 10A, and 10B are views illustrating an operation of an optical sensor of an electronic device according to various embodiments of the disclosure;

FIG. 11 is a view illustrating an operation of displaying a value measured by an optical sensor of an electronic device according to an embodiment of the disclosure; and

FIG. 12 is a flowchart illustrating a measurement operation of an optical sensor of an electronic device according to an embodiment of the disclosure.

The same reference numerals are used to represent the same elements throughout the drawings.

### MODE FOR THE INVENTION

The following description with reference to the accompanying drawings is provided to assist in a comprehensive understanding of various embodiments of the disclosure as defined by the claims and their equivalents. It includes various specific details to assist in that understanding, but these are to be regarded as merely exemplary. Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness.

The terms and words used in the following description and claims are not limited to the bibliographical meanings, but are merely used by the inventor to enable a clear and consistent understanding of the disclosure. Accordingly, it should be apparent to those skilled in the art that the following description of various embodiments of the disclosure is provided for illustration purposes only and not for the purpose of limiting the disclosure as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

It should be appreciated that the blocks in each flowchart and combinations of the flowcharts may be performed by one or more computer programs which include instructions. The entirety of the one or more computer programs may be stored in a single memory device or the one or more computer programs may be divided with different portions stored in different multiple memory devices.

Any of the functions or operations described herein can be processed by one processor or a combination of processors. The one processor or the combination of processors is circuitry performing processing and includes circuitry like an application processor (AP, e.g. a central processing unit (CPU)), a communication processor (CP, e.g., a modem), a graphics processing unit (GPU), a neural processing unit (NPU) (e.g., an artificial intelligence (AI) chip), a Wi-Fi chip, a Bluetooth® chip, a global positioning system (GPS) chip, a near field communication (NFC) chip, connectivity chips, a sensor controller, a touch controller, a finger-print sensor controller, a display driver integrated circuit (IC), an audio CODEC chip, a universal serial bus (USB) controller, a camera controller, an image processing IC, a microprocessor unit (MPU), a system on chip (SoC), an IC, or the like.

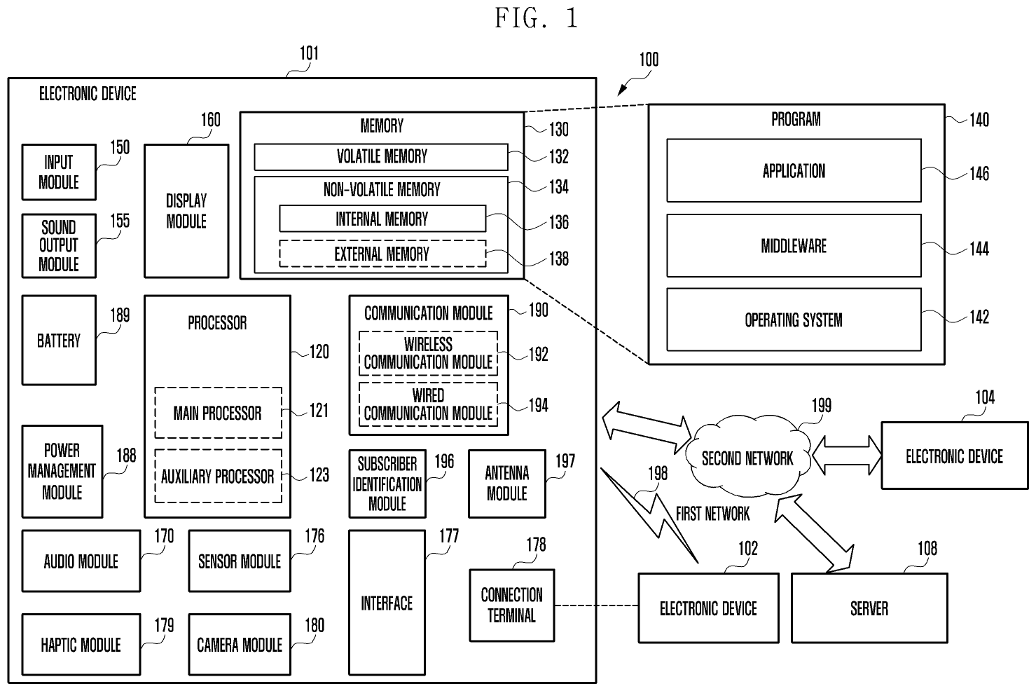

FIG. 1 is a block diagram illustrating an electronic device 101 in a network environment 100 according to an embodiment of the disclosure.

Referring to FIG. 1, the electronic device 101 in the network environment 100 may communicate with an electronic device 102 via a first network 198 (e.g., a short-range wireless communication network), or at least one of an electronic device 104 or a server 108 via a second network 199 (e.g., a long-range wireless communication network). According to an example, the electronic device 101 may communicate with the electronic device 104 via the server 108. According to an example, the electronic device 101 may include a processor 120, memory 130, an input module 150, a sound output module 155, a display module 160, an audio module 170, a sensor module 176, an interface 177, a connection terminal 178, a haptic module 179, a camera module 180, a power management module 188, a battery 189, a communication module 190, a subscriber identification module (SIM) 196, or an antenna module 197. In some examples, at least one of the components (e.g., the connection terminal 178) may be omitted from the electronic device 101, or one or more other components may be added in the electronic device 101. In some examples, some of the components (e.g., the sensor module 176, the camera module 180, or the antenna module 197) may be implemented as a single component (e.g., the display module 160).

The processor 120 may execute, for example, software (e.g., a program 140) to control at least one other component (e.g., a hardware or software component) of the electronic device 101 coupled with the processor 120, and may perform various data processing or computation. According to one example, as at least part of the data processing or computation, the processor 120 may store a command or data received from another component (e.g., the sensor module 176 or the communication module 190) in volatile memory 132, process the command or the data stored in the volatile memory 132, and store resulting data in non-volatile memory 134. According to an example, the processor 120 may include a main processor 121 (e.g., a central processing unit (CPU) or an application processor (AP)), or an auxiliary processor 123 (e.g., a graphics processing unit (GPU), a neural processing unit (NPU), an image signal processor (ISP), a sensor hub processor, or a communication processor (CP)) that is operable independently from, or in conjunction with, the main processor 121. For example, when the electronic device 101 includes the main processor 121 and the auxiliary processor 123, the auxiliary processor 123 may be adapted to consume less power than the main processor 121, or to be specific to a specified function. The auxiliary processor 123 may be implemented as separate from, or as part of the main processor 121.

The auxiliary processor 123 may control at least some of functions or states related to at least one component (e.g., the display module 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., executing an application). According to an example, the auxiliary processor 123 (e.g., an image signal processor or a communication processor) may be implemented as part of another component (e.g., the camera module 180 or the communication module 190) functionally related to the auxiliary processor 123. According to an example, the auxiliary processor 123 (e.g., the neural processing unit) may include a hardware structure specified for artificial intelligence model processing. An artificial intelligence model may be generated by machine learning. Such learning may be performed, e.g., by the electronic device 101 where the artificial intelligence is performed or via a separate server (e.g., the server 108). Learning algorithms may include, but are not limited to, e.g., supervised learning, unsupervised learning, semi-supervised learning, or reinforcement learning. The artificial intelligence model may include a plurality of artificial neural network layers. The artificial neural network may be a deep neural network (DNN), a convolutional neural network (CNN), a recurrent neural network (RNN), a restricted boltzmann machine (RBM), a deep belief network (DBN), a bidirectional recurrent deep neural network (BRDNN), deep Q-network or a combination of two or more thereof but is not limited thereto. The artificial intelligence model may, additionally or alternatively, include a software structure other than the hardware structure.

The memory 130 may store various data used by at least one component (e.g., the processor 120 or the sensor module 176) of the electronic device 101. The various data may include, for example, software (e.g., the program 140) and input data or output data for a command related thereto. The memory 130 may include the volatile memory 132 or the non-volatile memory 134. The non-volatile memory may include at least one of an internal memory 136 and an external memory 138.

The program 140 may be stored in the memory 130 as software, and may include, for example, an operating system (OS) 142, middleware 144, or an application 146.

The input module 150 may receive a command or data to be used by another component (e.g., the processor 120) of the electronic device 101, from the outside (e.g., a user) of the electronic device 101. The input module 150 may include, for example, a microphone, a mouse, a keyboard, a key (e.g., a button), or a digital pen (e.g., a stylus pen).

The sound output module 155 may output sound signals to the outside of the electronic device 101. The sound output module 155 may include, for example, a speaker or a receiver. The speaker may be used for general purposes, such as playing multimedia or playing record. The receiver may be used for receiving incoming calls. According to an example, the receiver may be implemented as separate from, or as part of the speaker.

The display module 160 may visually provide information to the outside (e.g., a user) of the electronic device 101. The display module 160 may include, for example, a display, a hologram device, or a projector and control circuitry to control a corresponding one of the display, hologram device, and projector. According to an example, the display module 160 may include a touch sensor adapted to detect a touch, or a pressure sensor adapted to measure the intensity of force incurred by the touch.

The audio module 170 may convert a sound into an electrical signal and vice versa. According to an example, the audio module 170 may obtain the sound via the input module 150, or output the sound via the sound output module 155 or a headphone of an external electronic device (e.g., the electronic device 102) directly (e.g., wiredly) or wirelessly coupled with the electronic device 101.

The sensor module 176 may detect an operational state (e.g., power or temperature) of the electronic device 101 or an environmental state (e.g., a state of a user) external to the electronic device 101, and then generate an electrical signal or data value corresponding to the detected state. According to an example, the sensor module 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. According to an example, the interface 177 may include, for example, a high definition multimedia interface (HDMI), a universal serial bus (USB) interface, a secure digital (SD) card interface, or an audio interface.

The connection terminal 178 may include a connector via which the electronic device 101 may be physically connected with the external electronic device (e.g., the electronic device 102). According to an example, the connection terminal 178 may include, for example, an HDMI connector, a USB connector, an SD card connector, or an audio connector (e.g., a headphone connector).

The haptic module 179 may convert an electrical signal into a mechanical stimulus (e.g., a vibration or a movement) or electrical stimulus which may be recognized by a user via his tactile sensation or kinesthetic sensation. According to an example, the haptic module 179 may include, for example, a motor, a piezoelectric element, or an electric stimulator.

The camera module 180 may capture a still image or moving images. According to an example, the camera module 180 may include one or more lenses, image sensors, image signal processors, or flashes.

The power management module 188 may manage power supplied to the electronic device 101. According to one example, the power management module 188 may be implemented as at least part of, for example, a power management integrated circuit (PMIC).

The battery 189 may supply power to at least one component of the electronic device 101. According to an example, the battery 189 may include, for example, a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a direct (e.g., wired) communication or a wireless communication. According to an example, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device via the first network 198 (e.g., a short-range communication network, such as Bluetooth™, wireless-fidelity (Wi-Fi) direct, or infrared data association (IrDA)) or the second network 199 (e.g., a long-range communication network, such as a legacy cellular network, a fifth generation (5G) network, a next-generation communication network, the Internet, or a computer network (e.g., LAN or wide area network (WAN)). These various types of communication modules may be implemented as a single component (e.g., a single chip), or may be implemented as multi components (e.g., multi chips) separate from each other. The wireless communication module 192 may identify and authenticate the electronic device 101 in a communication network, such as the first network 198 or the second network 199, using subscriber information (e.g., international mobile subscriber identity (IMSI)) stored in the subscriber identification module 196.

The wireless communication module 192 may support a 5G network, after a fourth generation (4G) network, and next-generation communication technology, e.g., new radio (NR) access technology. The NR access technology may support enhanced mobile broadband (eMBB), massive machine type communications (mMTC), or ultra-reliable and low-latency communications (URLLC). The wireless communication module 192 may support a high-frequency band (e.g., the millimeter wave (mm Wave) band) to achieve, e.g., a high data transmission rate. The wireless communication module 192 may support various technologies for securing performance on a high-frequency band, such as, e.g., beamforming, massive multiple-input and multiple-output (massive MIMO), full dimensional MIMO (FD-MIMO), array antenna, analog beam-forming, or large scale antenna. The wireless communication module 192 may support various requirements specified in the electronic device 101, an external electronic device (e.g., the electronic device 104), or a network system (e.g., the second network 199). According to an example, the wireless communication module 192 may support a peak data rate (e.g., 20 gigabits per second (Gbps) or more) for implementing eMBB, loss coverage (e.g., 164 decibels (dB) or less) for implementing mMTC, or U-plane latency (e.g., 0.5 milliseconds (ms) or less for each of downlink (DL) and uplink (UL), or a round trip of Ims or less) for implementing URLLC.

The antenna module 197 may transmit or receive a signal or power to or from the outside (e.g., the external electronic device) of the electronic device 101. According to an example, the antenna module 197 may include an antenna including a radiating element composed of a conductive material or a conductive pattern formed in or on a substrate (e.g., a printed circuit board (PCB)). According to an example, the antenna module 197 may include a plurality of antennas (e.g., array antennas). In such a case, at least one antenna appropriate for a communication scheme used in the communication network, such as the first network 198 or the second network 199, may be selected, for example, by the communication module 190 (e.g., the wireless communication module 192) from the plurality of antennas. The signal or the power may then be transmitted or received between the communication module 190 and the external electronic device via the selected at least one antenna. According to an example, another component (e.g., a radio frequency integrated circuit (RFIC)) other than the radiating element may be additionally formed as part of the antenna module 197.

According to various examples, the antenna module 197 may form an mm Wave antenna module. According to an example, the mm Wave antenna module may include a printed circuit board, an RFIC disposed on a first surface (e.g., the bottom surface) of the printed circuit board, or adjacent to the first surface and capable of supporting a designated high-frequency band (e.g., the mm Wave band), and a plurality of antennas (e.g., array antennas) disposed on a second surface (e.g., the top or a side surface) of the printed circuit board, or adjacent to the second surface and capable of transmitting or receiving signals of the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an example, commands or data may be transmitted or received between the electronic device 101 and the external electronic device 104 via the server 108 coupled with the second network 199. Each of the electronic devices 102 or 104 may be a device of a same type as, or a different type, from the electronic device 101. According to an example, all or some of operations to be executed at the electronic device 101 may be executed at one or more of the external electronic devices (e.g. electronic devices 102 and 104 or the server 108). For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, mobile edge computing (MEC), or client-server computing technology may be used, for example. The electronic device 101 may provide ultra low-latency services using, e.g., distributed computing or mobile edge computing. In an example, the external electronic device 104 may include an internet-of-things (IoT) device. The server 108 may be an intelligent server using machine learning and/or a neural network. According to an example, the external electronic device 104 or the server 108 may be included in the second network 199. The electronic device 101 may be applied to intelligent services (e.g., smart home, smart city, smart car, or healthcare) based on 5G communication technology or IoT-related technology.

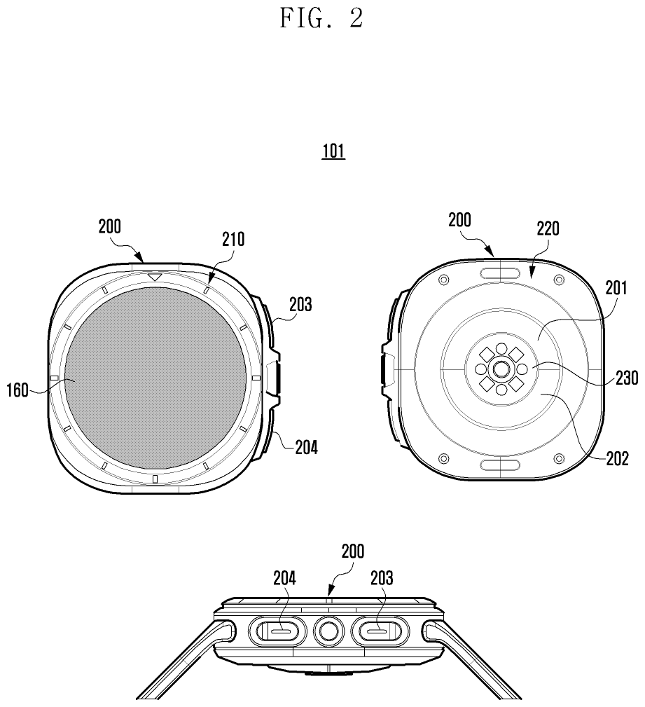

FIG. 2 is a perspective diagram illustrating a plane or cross section of a wearable electronic device (e.g., the electronic device 101 in FIG. 1) according to an embodiment of the disclosure.

Referring to FIG. 2, a housing 200 of the electronic device 101 (e.g., a wearable electronic device or a wearable watch) may be detachably worn on a body portion (e.g., the wrist or ankle) of the user.

The housing 200 may include a first surface (e.g., a front surface) 210 on which a display is disposed, a second surface (e.g., a rear surface) 220 coming in contact with at least a portion of the body when worn, and a third surface (e.g., a lateral surface) disposed to surround a space between the front surface 210 and the rear surface 220. The housing 200 may be implemented to have various shapes such as a circle, an oval, a square, and a round square. At least a portion of the first surface 210 of the housing 200 may be configured by a substantially transparent front plate (e.g., a glass plate including various coating layers or polymer plate). The second surface 220 may be configured by a rear plate and may be may of, for example, coated or colored glass, ceramic, polymers, metals (e.g., aluminum, stainless steel (STS), titanium, or magnesium), or a combination of at least two thereof. At least a portion of the rear plate may include a transparent cover and light emitted from a biometric sensor 230 may be emitted to the outside of the electronic device through the transparent cover. The lateral surface may be coupled to the front plate and the rear plate and may be configured by a lateral bezel structure including a metal and/or polymer. The rear plate and the lateral bezel structure may be integrally configured and include the same material (e.g., a metal material such as aluminum). An exterior of the housing 200 may be made of various materials, such as titanium, stainless steel, aluminum, or ceramic, which may withstand external impacts and scratches while also providing distinctive design features.

The housing 200 may include an input device that may be configured in the form of a physical button and/or crown for operating the electronic device 101 on the lateral surface and may be implemented to operate by detecting various gestural actions such as pressure, touch, proximity, or rotation. Additionally, the lateral surface of the housing may include a sensor capable of detecting touch, pressure, or gestures, either as a replacement for or in addition to the physical button and/or the crown, to recognize input actions.

The button may be made of a metal through which an electrical signal passes, and accordingly, the button may be implemented as an interface of an electrode sensor 203 or 204.

The biometric sensor 230 may be disposed on a printed circuit board disposed to face the second surface 220 of the housing 200. The biometric sensor 230 may include an optical sensor configured to emit light to a living body and receive the light that has been absorbed, scattered, and/or reflected. An emitter of the optical sensor may include multiple light-emitting elements to emit light of various bands and may include a device such as a light emitting diode (LED), a laser, and a vertical cavity surface emitting laser (VCSEL). The band of the light emitted from the emitter may include various wavelengths such as green, red, infrared (IR), blue, yellow, and ultraviolet (UV). Hereinafter, the biometric sensor may be referred to as a biometric sensor or an optical sensor.

A receiver of the biometric sensor 230 may include multiple light-receiving elements to receive reflected or transmitted light emitted from the emitter and may cause a value having been converted through an analog to digital converter (ADC) to be stored in a memory or a sensor buffer. The receiver may include a photodiode (PD) and/or complementary metal oxide semiconductor (CMOS) image sensor. The receiver may include a filter and accordingly, may receive light in a predetermined band or filter light outside a predetermined band.

The biometric sensor 230 may include a sensor control unit. The sensor control unit may be implemented as an IC and/or an analog front-end (AFE), control the emitter and the receiver of the optical sensor 230, process received data, and transmit processed data to a processor or store same in memory. Meanwhile, in addition to the optical sensor, the biometric sensor 230 may include an acoustic sensor that emits sound waves instead of light to detect a target. The biometric sensor may be implemented as a combination of various sensors, such as an optical sensor that emits light and receives transmitted, absorbed, scattered, and/or reflected light, an acoustic sensor that emits sound waves and receives reflected sound waves, and/or an image sensor for capturing images.

The biometric sensor 230 may include an optical sensor (e.g., a PPG sensor) configured to detect pulse waves with light and measure biometric information including heart rate (HR), heart rate variability (HRV), blood oxygen (SpO2), and blood pressure. The biometric sensor 230 may include a biomarker sensor configured to detect a specific substance or component in the body. A biomarker serves as an indicator of changes within the body, such as cells, blood vessels, proteins, deoxyribonucleic acid (DNA), ribonucleic acid (RNA), or metabolites, and information related to blood sugar, alcohol, advanced glycation end-products (AGEs), and antioxidants may be detected.

The AGEs are fats or proteins bonded with sugar and these substances are associated with aging and are known to exacerbate the progression of degenerative diseases such as diabetes, atherosclerosis, chronic kidney disease, and Alzheimer's disease. By using the optical sensor 230 to measure AGEs, it is possible to assess the user's current level of aging-related substance production or accumulation and provide information to help prevent same. Accordingly, various solutions may be provided to help users live a healthy life.

AGE sensing, which involves measuring advanced glycation end-products in the skin, may be applied to various electronic devices designed to allow a sensor to make contact with the skin. For example, the AGE sensing may be applied to various electronic devices, such as wrist-worn watches, bendable devices, band-type devices, or attachable devices affixed to the body, enabling continuous monitoring.

While the following embodiments mainly describe a wearable electronic device, an optical sensor according to various embodiments may be provided in various electronic devices such as the back of smartphones or tablets, dedicated sensing devices, or smartphone cover devices so as to be implemented to perform measurement by placing a palm of a hand thereon.

According to an embodiment of the disclosure, the optical sensor 230 may be disposed to face the second surface 220 (e.g., the rear surface that contacts the user's body) of the housing (e.g., the housing 200 in FIG. 2).

The emitter of the optical sensor 230 may emit light in various bands. For example, the emitter may include elements such as LED, laser, and VCSEL.

The electronic device 101 may include a temperature sensor, for example, inside the second surface 220. The temperature sensor may measure a temperature of an organism or component and may be implemented as contact or non-contact depending on methods. A temperature value measured by the temperature sensor may be stored in the memory or transferred to the processor to be used for skin temperature estimation or used for situation recognition and/or body temperature estimation.

The electronic device 101 may include an electrode sensor including a first electrode 201, a second electrode 202, a third electrode 203, and/or a fourth electrode 204. The electrode sensor may be implemented as a sensor having an electrode configured to detect a characteristic of a living body through contact with the living body. The electrode serves as an interface for measuring the electrical characteristics (a voltage, current, or impedance) of the user's body, enabling configuration of an equivalent circuit via the body to detect various physiological characteristics.

The electronic device 101 may measure physiological conditions by detecting electrical signals generated from bodily activities, such as electrocardiograms (ECG), electromyograms (EMG), and/or electroencephalograms (EEG), through the electrode sensor. For example, the electronic device 101 may use an INP electrode (e.g., the first electrode 201 and the second electrode 202) (the wrist) to measure the ECG and an INM electrode (e.g., the third electrode 203 and the fourth electrode 204) (the opposite finger) to measure an electrical signal generated from the heart. In this case, the accuracy of biometric signal measurement may be increased by matching the potential reference of a biometric signal by grounding an RLD electrode.

The electronic device 101 may use four electrodes to measure a body composition through body impedance analysis (BIA) measurement. The body composition may include information such as body fat, body water, and musculoskeletal mass. The electronic device 101 may use two electrodes to measure an electrodermal activity (EDA) of the skin. The EDA may include measurements related to electrical skin response, such as skin conductance, a galvanic skin response (GSR), an electrodermal response (EDR), and a psychogalvanic reflex (PRG).

The electronic device 101 may measure various biometric indexes and generate biometric data based on a sensor circuit connected to the body through an electrode interface.

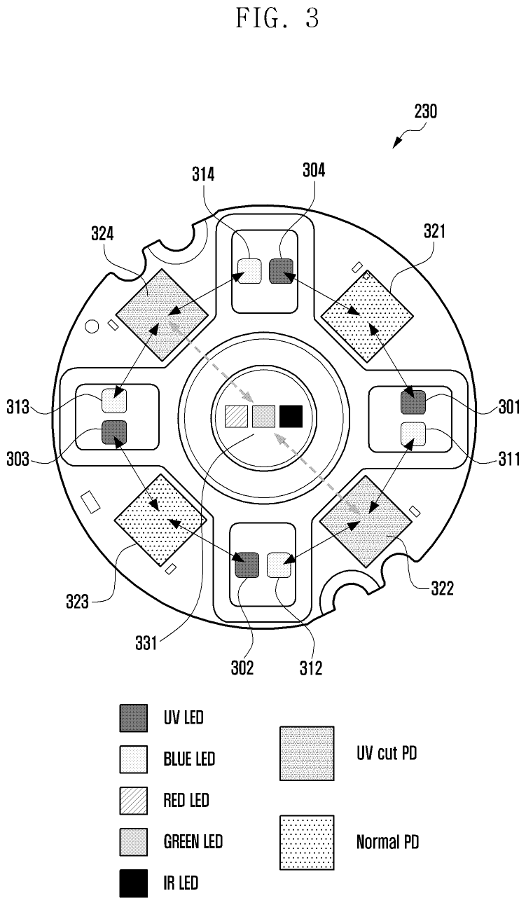

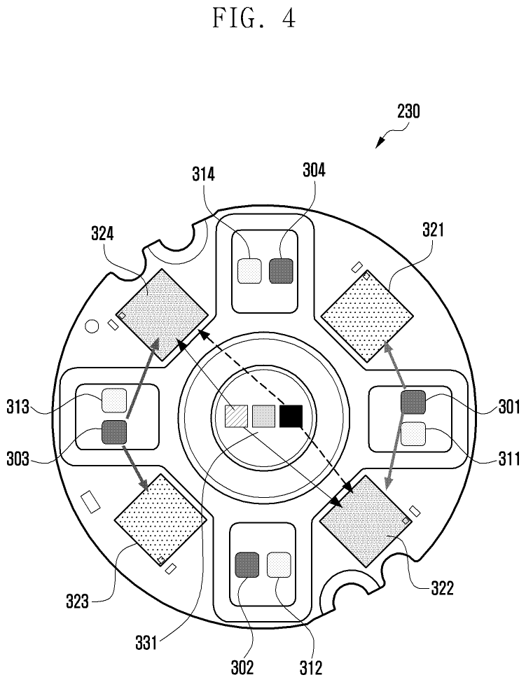

FIGS. 3 and 4 are views illustrating an optical sensor (e.g., the optical sensor 230 in FIG. 2) of an electronic device (e.g., the electronic device 101 in FIG. 1 or 2) according to various embodiments of the disclosure.

Referring to FIGS. 3 and 4, the optical sensor 230 may include an emitter including multiple light-emitting elements and a receiver including multiple light-receiving elements.

The optical sensor 230 may include the multiple light-emitting elements configured to emit light of various wavelengths, such as green, red, infrared (IR), blue, violet, yellow, and ultraviolet (UV).

A light-emitting element 301, 302, 303 and/or 304 configured to emit light in a first band (e.g., an UV wavelength) may be disposed in a peripheral area of the printed circuit board of the optical sensor 230. Light-emitting elements 301, 302, 303 and/or 304 configured to emit light in the first band (e.g., the UV wavelength) may be disposed in positions symmetrical to each other. For example, the light-emitting elements 301, 302, 303 and/or 304 configured to emit light in the first band (e.g., the UV wavelength) may be arranged in areas corresponding to positions in direction of about 0 degrees (12 o'clock position), about 90 degrees (3 o'clock position), about 180 degrees (6 o'clock position), and about 270 degrees (9 o'clock position), respectively, on the second surface of the housing along the peripheral area based on the center of the printed circuit board or the optical sensor 230.

The light-emitting elements 301, 302, 303 and/or 304 configured to emit light in the first band (e.g., the UV wavelength) may arranged spaced apart from light-emitting elements disposed at another position and/or light-emitting elements configured to emit light in a band different from the first band. For example, the light-emitting elements 301, 302, 303 and/or 304 configured to emit light in the first band (e.g., the UV wavelength) may additionally employ a partition wall structure for optical isolation so as to be separated from light emitted from light-emitting elements disposed at another position and/or light-emitting elements configured to emit light in a band different from the first band.

The electronic device 101 may use the light-emitting elements 301, 302, 303 and/or 304 configured to emit light in the first band (e.g., the UV wavelength) to measure advanced glycation end-products (AGEs).

Light-emitting elements 311, 312, 313 and/or 314 configured to emit light in a second band (e.g., a blue wavelength) may be selectively mounted together with each of the light-emitting elements 301, 302, 303, and/or 304 configured emit light in the first band (e.g., the UV wavelength), with each pair positioned in an outer area (e.g., at the 12 o'clock, 3 o'clock, 6 o'clock, and/or 9 o'clock positions).

Light-emitting elements (e.g., R (red), G (green), and/or IR wavelength) configured to emit light in a band different from the first band (e.g., the UV wavelength) may be arranged at a center area 331.

The receiver of the optical sensor 230 may receive light emitted from the emitter and transmitted, absorbed, scattered, and/or reflected.

The optical sensor 230 may include various light-receiving elements, such as a photodiode (PD) and/or complementary metal oxide semiconductor (CMOS) image sensor. The light-receiving elements may include a filter and accordingly, may receive light in a predetermined band or filter light in a predetermined band.

The optical sensor 230 may include multiple light-receiving elements. The multiple light-receiving elements may have different reactivities. For example, the multiple light-receiving elements may include a light-receiving element capable of receiving light in a predetermined band, a light-receiving element capable of filtering light in a predetermined band and receiving light in another band, and/or a light-receiving element capable of receiving light in all bands. For example, the optical sensor 230 may include a first light-receiving element (e.g., a normal PD) capable of receiving light in all bands and/or a second light-receiving element (e.g., an UV cut PD or greed PD) capable of filtering the first band (e.g., the UV wavelength) and receiving light in another band. Hereinafter, the first light-receiving element and the second light-receiving element are explained as examples, but the embodiments are not limited thereto, and light-receiving elements having various reactivities may be applied.

The light-receiving elements may be arranged adjacent to but spaced a predetermined distance apart from the light-emitting elements in an outer area of the optical sensor 230.

The first light-receiving element 321 and the second light-receiving element 322 may be arranged adjacent to and spaced a predetermined distance apart from the outer area on which a first light-emitting element 301, 302, 303, or 304 is mounted. For example, the first light-receiving element 321 and the second light-receiving element 322 each may be arranged to be adjacent to the first light-emitting element 301. In this case, the first light-receiving element 321 and the second light-receiving element 322 may be arranged to be symmetrically spaced an identical distance apart from the outer area on which the first light-emitting element 301 is disposed. For example, the first light-receiving element 321 and the second light-receiving element 322 may be arranged to be spaced an identical or substantially identical distance (e.g., 3-3.5 mm) apart to the left (top) and right (bottom), respectively, in a clockwise direction from the outer area on which the first light-emitting element 301 is disposed.

The third light-receiving element 323 and the fourth light-receiving element 324 may be arranged adjacent to and spaced a predetermined distance apart from the outer area on which a third light-emitting element 303 is mounted. The third light-receiving element 323 may correspond to a light-receiving element (e.g., the normal PD) configured to receive light in all bands. The fourth light-receiving element 324 may be a light-receiving element (e.g., the UV cut PD or green PD) capable of filtering the first band (e.g., the UV wavelength) and receiving light in other bands. For example, the third light-receiving element 323 and the fourth light-receiving element 324 each may be arranged to be adjacent to the third light-emitting element 303. In this case, the third light-receiving element 323 and the fourth light-receiving element 324 may be arranged to be symmetrically spaced an identical distance apart from the outer area on which the third light-emitting element 303 is disposed. For example, the third light-receiving element 323 and the fourth light-receiving element 324 may be arranged to be spaced an identical or a substantially identical distance (e.g., 3-3.5 mm) apart to the left (top) and right (bottom), respectively, in a clockwise direction from the outer area on which the third light-emitting element 303 is disposed.

The multiple light-receiving elements including the first light-receiving element 321, the second light-receiving element 322, the third light-receiving element 323, and the fourth light-receiving element 324 may be arranged to be spaced an identical or substantially identical distance (e.g., 3-3.5 mm) apart from the center area 331 on which the multiple light-emitting elements are arranged. For example, the multiple light-receiving elements including the first light-receiving element 321, the second light-receiving element 322, the third light-receiving element 323, and the fourth light-receiving element 324 may be arranged in a circular shape along an outer edge so as to be spaced an identical or substantially identical distance (e.g., 3-3.5 mm) apart from each other along the outer area based on the center area 331 on which the multiple light-emitting elements are arranged.

The optical sensor 230 may receive light emitted from the multiple light-emitting elements (e.g., red, RI, and green LEDs) (the light-emitting elements arranged in the center area 331) through the second light-receiving elements 323 and 324 disposed in different areas at an identical or substantially identical distance to estimate a melanin index and through this, correct the AGE value.

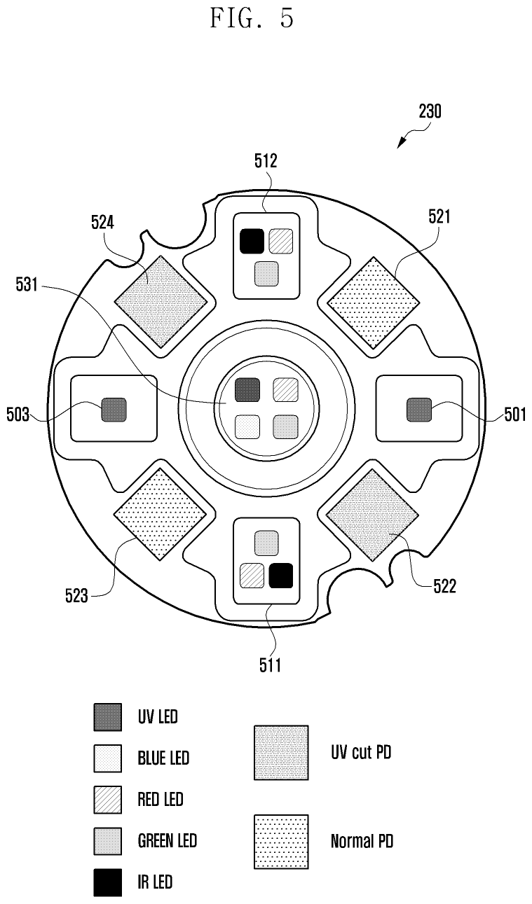

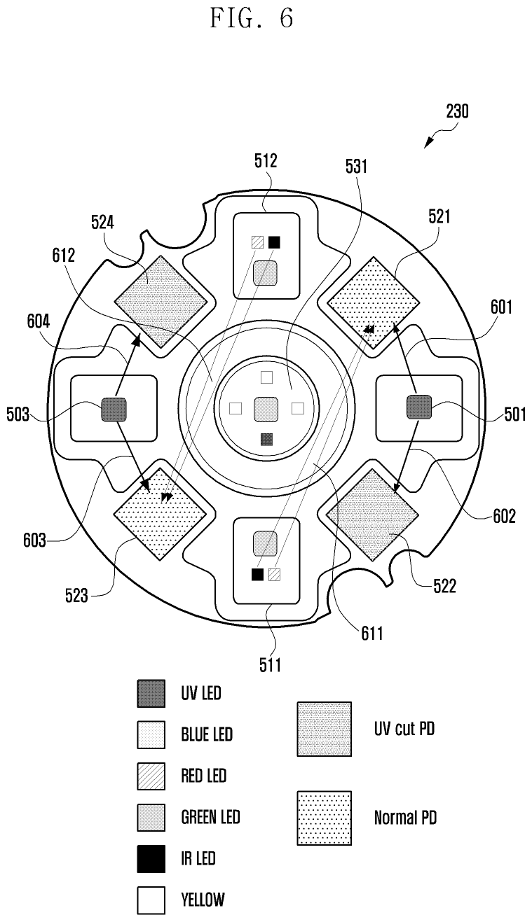

FIGS. 5 and 6 are views illustrating an optical sensor of an electronic device according to various embodiments of the disclosure.

Referring to FIGS. 5 and 6, the optical sensor 230 may include an emitter including multiple light-emitting elements and a receiver including multiple light-receiving elements.

The optical sensor 230 may be disposed on the printed circuit board to face the second surface 220 (e.g., the rear surface that contacts the user's body) of the housing (e.g., the housing 200 in FIG. 2).

The emitter of the optical sensor 230 may emit light in various bands. For example, the emitter may include elements such as LED, laser, and VCSEL.

The optical sensor 230 may include the multiple light-emitting elements configured to emit light of various wavelengths, such as green, red, infrared (IR), blue, yellow, and ultraviolet (UV).

A light-emitting element 501 and/or 503 configured to emit light in the first band (e.g., the UV wavelength) may be disposed in a peripheral area of the optical sensor 230. The light-emitting elements 501 and/or 503 configured to emit light in the first band (e.g., the UV wavelength) may be disposed in positions symmetrical to each other. For example, the light-emitting elements 501 and/or 503 configured to emit light in the first band (e.g., the UV wavelength) may be arranged in areas corresponding to positions in a direction of about 90 degrees (3 o'clock position) and about 270 degrees (9 o'clock position), respectively along the peripheral area based on the center area of the optical sensor 230 on the printed circuit board.

The light-emitting elements 501 and/or 503 configured to emit light in the first band (e.g., the UV wavelength) may be arranged spaced apart from light-emitting elements disposed at another position and/or light-emitting elements configured to emit light in a band different from the first band. For example, the light-emitting elements 501 and/or 503 configured to emit light in the first band (e.g., the UV wavelength) may additionally employ a partition wall structure so as to be separated from light emitted from light-emitting elements disposed at another position and/or light-emitting elements configured to emit light in a band different from the first band. A first area on which the first light-emitting element 501 configured to emit light in the first band is disposed and a second area on which the third light-emitting element 503 configured to emit light in the first band is disposed may correspond to positions which are substantially point symmetrical to each other with respect to the center area of the printed circuit board disposed to face the second surface of the housing in which the optical sensor 230 is disposed.

The electronic device 101 may use the light-emitting elements 501 and/or 503 configured to emit light in the first band (e.g., the UV wavelength) to measure the advanced glycation end-products (AGEs).

Light-emitting elements configured to emit light in a band different from the first band (e.g., the UV wavelength) (e.g., a R (red), G (green), B (blue), V (violet), Y (yellow), and/or IR (infrared) wavelength) may be disposed in the center area 531 and/or a peripheral area (a third area 511) and/or a fourth area 512) of the printed circuit board. The light-emitting elements (e.g., the R, G, B, V, Y and/or IR wavelength) configured to emit light in a band different from the first band (e.g., the UV wavelength) may be disposed in a peripheral area 511 and/or 512 separated and spaced apart from the area on which the light-emitting element 501 and/or 503 configured to emit light in the first band (e.g., the UV wavelength). For example, light-emitting elements configured to emit light in a specific band (e.g., a B, V, Y and/or IR wavelength) may be arranged in the center area 531. For example, light-emitting elements configured to emit light in a specific band (e.g., an R, G and/or IR wavelength) may be arranged at positions symmetrical to each other within the third area and the fourth area. For example, light-emitting elements configured to emit light in a specific band (e.g., a R, G and/or IR wavelength) may be arranged at the outer 12 o'clock position and 6 o'clock position of the optical sensor 230 to have a shape symmetrical to each other within the third area 511 and the fourth area 512. For example, the light-emitting elements configured to emit light in a specific band arranged in the third area 511 and the fourth area 512 may be arranged at positions substantially point symmetrical to each other with respect to the center area of the printed circuit board facing the second surface of the housing. The first area on which the first light-emitting element 501 configured to emit light in the first band is disposed, the second area on which the second light-emitting element 503 configured to emit light in the first band is disposed, and the third area 511 and the fourth area 512 on which the light-emitting elements (e.g., the R, G, B, V, Y and/or IR wavelengths) configured to emit light in the other bands are disposed may correspond to positions point symmetrical to each other with respect to the center portion of the printed circuit board facing the second surface of the housing on which the optical sensor 230 is disposed.

The receiver of the optical sensor 230 may receive light emitted from the emitter and transmitted, absorbed, scattered, and/or reflected.

The optical sensor 230 may include multiple light-receiving elements. The light-receiving elements may include various light-receiving elements, such as a photodiode (PD) and/or complementary metal oxide semiconductor (CMOS) image sensor. The light-receiving elements may include a filter and accordingly, may receive light in a predetermined band or filter light in a predetermined band.

The multiple light-receiving elements may have different reactivities. For example, the multiple light-receiving elements may include a light-receiving element capable of receiving light in a predetermined band, a light-receiving element capable of filtering light in a predetermined band and receiving light in another band, and/or a light-receiving element capable of receiving light in all bands. For example, the optical sensor 230 may include a first light-receiving element (e.g., a normal PD) capable of receiving light in all bands and/or a second light-receiving element (e.g., an UV cut PD or greed PD) configured to partially filter the first band (e.g., the UV wavelength) and receiving light in another band. Hereinafter, the first light-receiving element and the second light-receiving element are explained as examples, but the embodiments are not limited thereto, and light-receiving elements having various reactivities may be applied.

The light-receiving elements may be arranged adjacent to but spaced a predetermined distance apart from the light-emitting elements in an outer area of the optical sensor 230.

The first light-receiving element 521 and the second light-receiving element 522 each may be arranged adjacent to and spaced a predetermined distance apart from the outer area on which a first light-emitting element 501 is mounted. The first light-receiving element 521 may correspond to a light-receiving element (e.g., the normal PD) configured to receive light in all bands. The second light-receiving element 522 may be a light-receiving element (e.g., the UV cut PD or green PD) capable of filtering the first band (e.g., the UV wavelength) and receiving light in other bands. For example, the first light-receiving element 521 and the second light-receiving element 522 each may be arranged to be adjacent to the first light-emitting element 501. In this case, the first light-receiving element 521 and the second light-receiving element 522 may be arranged to be symmetrically to each other with respect to the outer area on which the first light-emitting element 501 is disposed. For example, the first light-receiving element 521 and the second light-receiving element 522 may be arranged to be spaced an identical or substantially identical distance (e.g., 3-3.5 mm) apart to the left (top) and right (bottom), respectively, in a clockwise direction from the outer area on which the first light-emitting element 501 is disposed. In addition, the third light-receiving element 523 and the fourth light-receiving element 524 may be arranged to be symmetrically spaced an identical distance apart from the outer area on which the third light-emitting element 503 is disposed. For example, the third light-receiving element 523 and the fourth light-receiving element 524 may be arranged to be spaced an identical or a substantially identical distance (e.g., 3-3.5 mm) apart to the left (bottom) and right (top), respectively, in a clockwise direction from the outer area on which the third light-emitting element 503 is disposed.

A first path 601 and a second path 602 respectively corresponding to an optical path from the first light-emitting element 501 to the first light-receiving element 521 and to the second light-receiving element 522 may have an identical distance. In addition, a first path 603 and a second path 604 respectively corresponding to an optical path from the third light-emitting element 503 to the third light-receiving element 523 and to the fourth light-receiving element 524 may have an identical length.

The multiple light-receiving elements including the first light-receiving element 521, the second light-receiving element 522, the third light-receiving element 523, and the fourth light-receiving element 524 may be arranged to be spaced an identical or substantially identical distance (e.g., 3-3.5 mm) apart from the center area 531 on which the multiple light-emitting elements are arranged. The third light-receiving element 323 may correspond to a light-receiving element (e.g., the normal PD) configured to receive light in all bands. The fourth light-receiving element 324 may be a light-receiving element (e.g., the UV cut PD or green PD) capable of filtering the first band (e.g., the UV wavelength) and receiving light in other bands. For example, the multiple light-receiving elements including the first light-receiving element 521, the second light-receiving element 522, the third light-receiving element 523, and the fourth light-receiving element 524 may be arranged in a circular shape along an outer edge so as to be spaced an identical or substantially identical distance (e.g., 3-3.5 mm) apart from each other along the outer area based on the center area 531 on which the multiple light-emitting elements are arranged.

The optical sensor 230 may receive light emitted from the multiple light-emitting elements (e.g., red, RI, and green LEDs) (the light-emitting elements arranged in the outer area 511 or 512) through the first light-receiving elements 521 and the third light-receiving element 523 disposed in different areas at an identical or substantially identical distance to estimate a melanin index and perform correction on a value of the AGEs.

The optical sensor 230 may be configured to receive, through the first light-receiving element 521 and the third light-receiving element 523, light emitted from light-emitting elements arranged symmetrically in the outer areas 511 and 512 at positions symmetrical to each other, for example, at the outer 12 o'clock position and the 6 o'clock position. For example, it may be configured to receive, through the first light-receiving element 521, light emitted from light-emitting elements in the outer area 511 at the outer 6 o'clock position of the optical sensor 230. For example, it may be configured to receive, through the third light-receiving element 523, light emitted from light-emitting elements in the outer area 512 at the outer 12 o'clock position of the optical sensor 230. This arrangement can ensure that the distance of an optical path 611 and an optical path 612 is the same and, within feasible limits, maximized to a longer distance (e.g., 8 mm or more). Accordingly, light of relatively long wavelengths (e.g., red, green, and/or IR) is absorbed or transmitted through the body and settles on the light-receiving elements, and the deviation due to settling may be improved (robustness), and the X-talk effect caused by light of other wavelengths may be reduced. Accordingly, skin tone sensing accuracy may be improved, and the melanin index may be improved according to correction, so skin tone accuracy may be improved.

An amount of light reflected by light with relatively long wavelengths (e.g., red and/or IR) is measured to estimate skin tone according to the amount and correct the index value of the AGEs. In this case, an average reference value of the amount of light reflected by light with relatively long wavelengths (e.g., red and/or IR) and skin tone corresponding thereto may be stored in advance. Accordingly, it may be configured to control the optical sensor 230 to emit light with relatively long wavelengths (e.g., red and/or IR), compare the amount of reflected light with the reference value, and correct the index value of the AGEs. In this case, the melanin index may not be estimated separately.

The light-emitting elements 501 and/or 503 configured to emit light in the designated first wavelength band (e.g., the UV wavelength) are arranged along the outer area at 90 degrees (3 o'clock position) and 270 degrees (9 o'clock position) relative to the center of the optical sensor 230, and the optical paths to the first light-receiving element 521, the second light-receiving element 522, the third light-receiving element 523, and the fourth light-receiving element 524 adjacent to the light-emitting elements are configured to be substantially equal in length, thereby enabling more accurate measurement of intensity of fluorescent light generated due to UV light incident on the user's body and improving the accuracy of advanced glycation end-product (AGE) measurement.

Table 1 shows characteristics of the optical sensor 230 according to embodiments of the disclosure.

TABLE 1

| LED | | Photodiode | |
|---|---|---|---|
| color | center wavelength | type | coverage |
| UV | 365 nm | Normal PD | 340 nm-980 nm |
| Blue | 405 nm or 470 nm | UV cut PD | 480 nm-980 nm |
| Green | 525 nm | | |
| Red | 660 nm | | |
| IR | 940 nm | | |

In one embodiment, the optical sensor 230 may include the first light-emitting element, the first light-receiving element, and the second light-receiving element so as to have optical paths of equal length from the first light-emitting element (e.g., an UV LED) to the second light-receiving element (e.g., the UV cut PD) configured to receive fluorescent light generated when emitted light reaches the user's body and to the first light-receiving element (e.g., the normal PD) configured to receive UV light reflected from the user's body. The measurement value of the AGEs may change sensitively depending on the amount of light received by the first light-receiving element and the second light-receiving element. Therefore, by equalizing the length of the optical paths, the measurement value of the AGEs may be more accurate even when the magnitude of a signal changes somewhat differently due to wearing conditions. In addition, by disposing the first light-emitting element (e.g., the UV LED) to be spaced apart from the second light-emitting elements (e.g., a red, green, and/or IR LED), the influence of noise may be further prevented by ensuring that the emission of the first light-emitting element does not excite the second light-emitting elements or generate optical signals regardless of whether current is applied,

The first light-emitting element (e.g., the UV LED) may be mounted separately from LEDs of other wavelengths. By mounting the UV LED alone separately from other LEDs and arranging the optical path lengths to be equal for both the first light-receiving element (e.g., the normal PD) and the second light-receiving element (e.g., the UV cut PD), measurement value deviations due to wearing conditions may be further improved.

Among the multiple light-emitting devices, for example, R, G, B, and IR LEDs may all be used to measure skin tone and melanin. In the structure shown in FIG. 6, where multiple light-receiving elements are arranged, the emitted light from G (Green) and B (Blue) light-emitting elements, which have relatively shorter wavelengths, may be more effectively received by the light-receiving elements that are relatively closer to the corresponding light-emitting elements and the emitted light from R (Red) and IR (Infrared) light-emitting elements, which have relatively longer wavelengths, may be more effectively received by the light-receiving elements that are relatively farther from the corresponding light-emitting elements. Furthermore, multiple light-emitting elements, for example, light-emitting elements configured to emit R (Red), G (Green), and IR (Infrared) light, may be arranged in a multi-array configuration at symmetrically opposite positions in the outer regions, such as at the 12 o'clock position 512 and the 6 o'clock position 511 and the multiple light-emitting elements may be arranged to be point-symmetrical with respect to the center of the shape of the optical sensor 230.

The multiple light-emitting elements may be implemented as an element configured to emit laser-type light having improved optical characteristics, such as a vertical-cavity surface-emitting laser (VCSEL) and a laser diode (LD). The use of lasers may enable deeper body penetration and more precise biomarker detection, thereby improving performance.

At least a portion of the light-receiving elements may be replaced with an optical sensor capable of color picking to improve skin tone or melanin detection accuracy and an image sensor such as a CMOS may be disposed therefor.

The multiple light-receiving elements may include multiple PDs having different pixels capable of receiving light in multiple different bands. For example, PDs having different pixels capable of detecting light in red, green, blue, and clear (full spectrum) bands may be used to measure the light intensity for each band. In case of measuring using different types of PDs that measure light intensity for each band, it is possible to emit R, G, B, V, and IR light substantially simultaneously for measurement to perform measurement so as to allow for measurement with a higher sampling frequency.

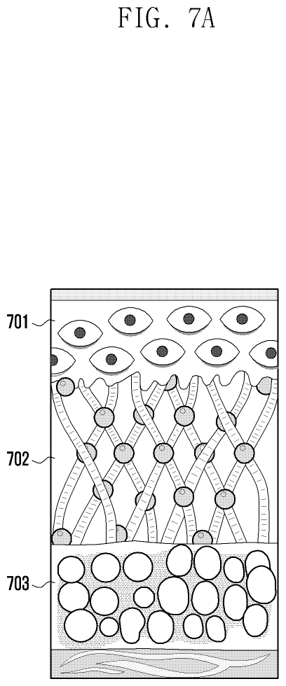

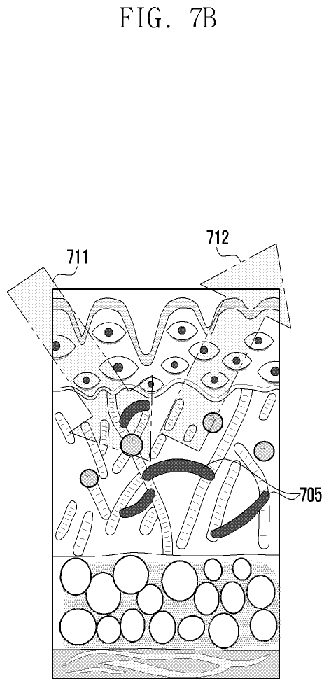

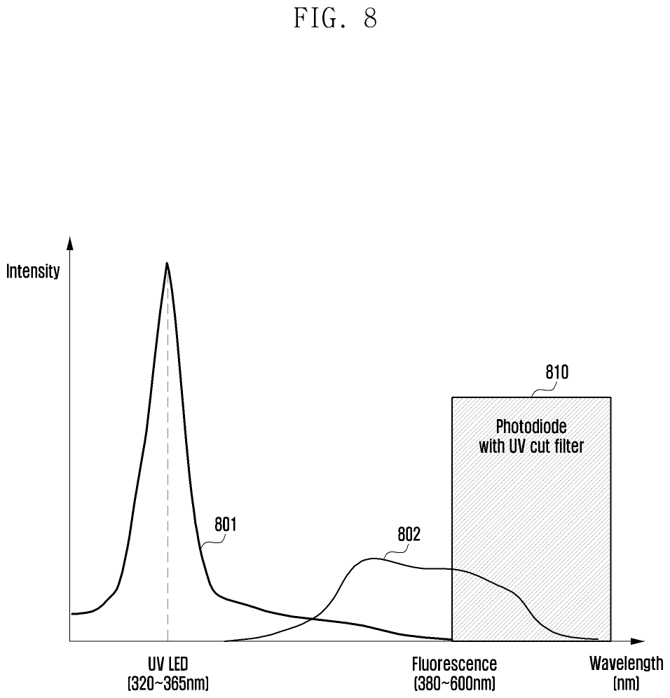

FIGS. 7A, 7B, and 8 are views illustrating an operation of an optical sensor (e.g., the optical sensor 230 in FIGS. 2 to 6) of an electronic device (e.g., the electronic device 101 in FIG. 1) according to various embodiments of the disclosure.

Referring to FIGS. 7A, 7B, and 8, the optical sensor 230 of the electronic device 101 may use one or more first light-emitting elements (e.g., the first light-emitting elements 301, 302, 303, 304, 501, and/or 503 in FIGS. 3 to 6) to measure advanced glycation end-products (AGEs).

Light 711 in a designated band (e.g., UV wavelength of about 320˜365 nm) emitted from the first light-emitting element may be emitted to the user's body surface. The emitted light may, for example, pass through the epidermis 701 of the user's skin, which includes the epidermis 701, dermis 702, and subcutaneous tissue 703 and then be absorbed by advanced glycation end-products (AGEs) 705 accumulated in the dermis 702, resulting in the emission of fluorescent light with a green spectrum (e.g., about 500 nm wavelength) 712, and the optical sensor 230 may receive and measure the fluorescent light.

The x-axis of FIG. 8 may represent the wavelength (nm) of light and the y-axis may represent the relative intensity of light. As shown in FIG. 8, UV light 801 emitted from the first light-emitting element may have a wavelength of about 320 to 365 nm, while the fluorescent light 802 with a green spectrum emitted when UV light is absorbed by advanced glycation end-products may have a wavelength of about 380 to 600 nm, allowing the second light-receiving element (e.g., the UV cut PD) equipped with a filter that excludes UV light to receive a wavelength band of the fluorescent light 810 while excluding the overlapping wavelength band of the UV light and the fluorescent light.

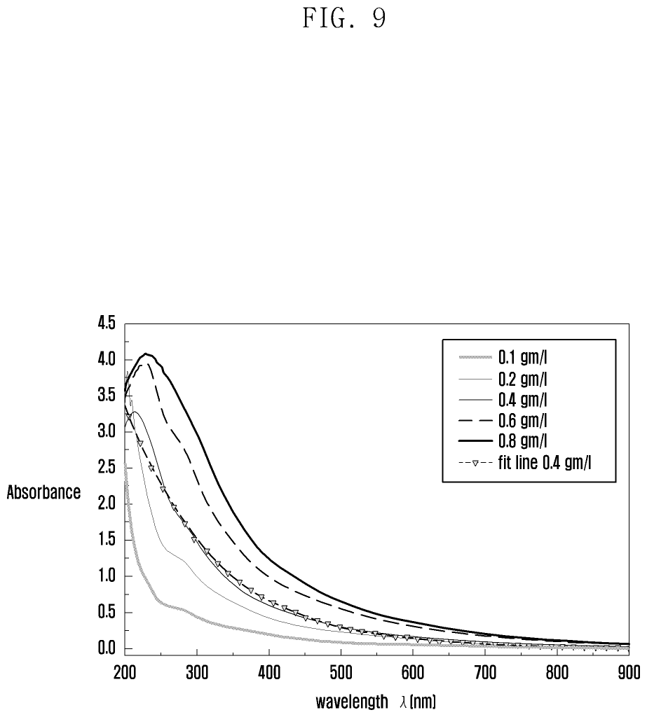

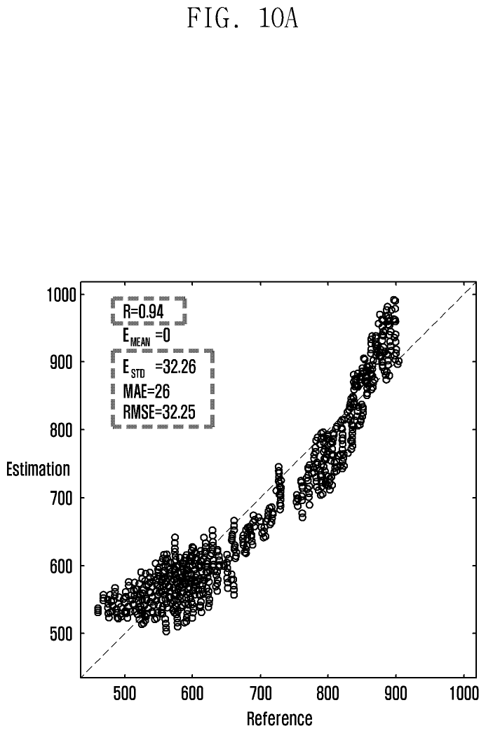

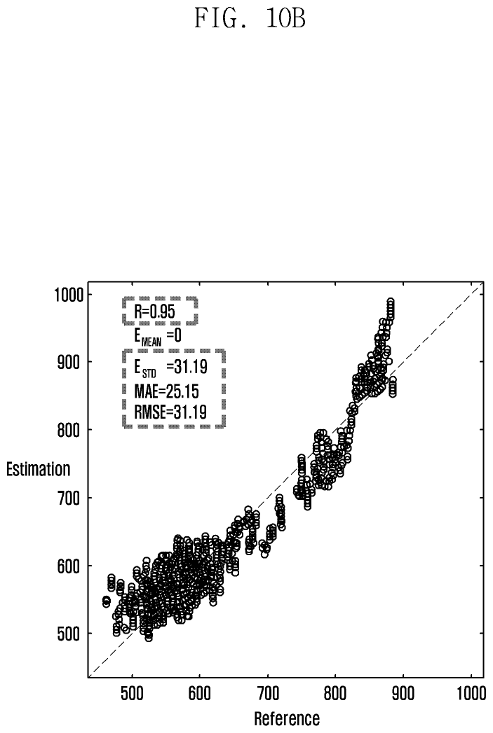

FIGS. 9, 10A, and 10B are views illustrating an operation of an optical sensor (e.g., the optical sensor 230 in FIGS. 2 to 6) of an electronic device (e.g., the electronic device 101 in FIG. 1 or 2) according to various embodiment of the disclosure.

Referring to FIGS. 9, 10A, and 10B, the UV light may be absorbed significantly by melanin, as shown in the melanin absorption spectrum. In FIG. 9, the x-axis may represent wavelength (nm), the y-axis may represent absorption rate (%), and graphs may show changes in absorption rate according to light wavelength for melanin concentrations of 0.1, 0.2, 0.4, 0.6, and 0.8 (gm/l) respectively. According to the drawing, it may be identified that the higher the melanin concentration, the higher the light absorption rate, and specifically, in the UV light wavelength band (about 300 nm), the higher the melanin concentration, the significantly higher the light absorption rate. Accordingly, the intensity of reflected UV light may vary depending on skin tone, specifically melanin concentration, and accurate measurement of advanced glycation end-products may require UV signal correction based on skin tone (melanin index) measurement. The melanin index may be indirectly calculated through an R, G, and B value by Equation 1 below. The equation is merely an example, and the structure and performance of a measurement sensor may vary depending on equations. Furthermore, an IR value may be additionally used in addition to the R, G, and B value.

$$melanin\_index = 0.31 ⋆ R + 0.58 ⋆ G + 0.11 ⋆ B \qquad Equation 1$$

Equation 1 above is merely an example for understanding and an embodiment of the disclosure may not be limited thereto. For example, Equation 1 above may be transformed, applied or expanded in various manners.

FIGS. 10A and 10B are views illustrating an effect of correcting a measurement value by an optical sensor according to an embodiment of the disclosure.

Referring to FIGS. 10A and 10B, for the accurate skin tone (melanin index) correction, the light-emitting elements (e.g., the red and IR LED) arranged in the outer area (e.g., the outer area 511 and 512 of the 12 o'clock position and the 6 o'clock position in FIG. 5 or 6) and the second light-receiving element (e.g., the normal PD) may be configured to have a longer optical path (e.g., 8 mm or longer). In addition, the light-emitting elements arranged in the outer area 511 and 512 of the 12 o'clock position and the 6 o'clock position may be arranged in a symmetrical structure having an identical optical path. Therefore, compared to the case of short optical path, the effect according to the wearing condition may be reduced. In addition, as the optical path becomes longer, the melanin sensing area expands, which may improve skin tone measurement accuracy.

In a stable state without movement, the x-axis represents melanin index values measured using specialized melanin measurement equipment, while the y-axis represents melanin index values measured using the optical sensor 230 of the electronic device 101 according to the length of the optical path, allowing for a comparison of the correlation therebetween. FIG. 10A shows the correlation of measurement values when the distance between the light-emitting element and the light-receiving element is relatively short, resulting in a shorter optical path, while FIG. 10B shows the correlation of measurement values when the distance between the light-emitting element and the light-receiving element is relatively long, resulting in a longer optical path. Compared to the shorter optical path in FIG. 10A, the longer optical path in FIG. 10B shows an improvement in skin correction performance, with the correlation increasing by approximately 0.1 from about 0.94 to about 0.95 and the error decreasing by about 3% from about 32.26 to about 31.19 in terms of standard variation (ESTD).

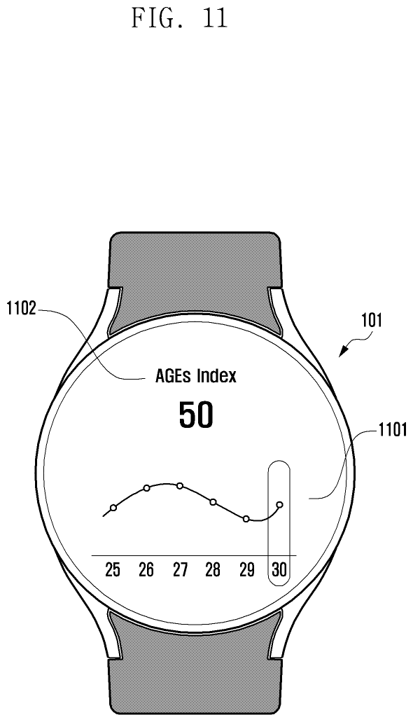

FIG. 11 is a view illustrating an operation of displaying a value measured by an optical sensor (e.g., the optical sensor 230 in FIGS. 2 to 6) of an electronic device (e.g., the electronic device 101 in FIG. 1 or 2) according to an embodiment of the disclosure.

Referring to FIG. 11, the electronic device 101 may measure an amount of fluorescent light (green) detected by multiple light-receiving elements to measure the AGE value measured by the biometric sensor or the optical sensor 230, and may estimate a modeled AGE index 1102 value according to the size of the amount of light.

A higher amount of AGEs may result in a lower AGE index 1102 value, and a lower amount of AGEs may result in a higher AGE index 1102 value. However, the index value for AGE may also be defined in the opposite way.

The electronic device 101 may be configured to measure the AGE value every day (daily unit) and display the AGE index 1102 value on a display 1101 (e.g., the display module 160 in FIG. 1).

The electronic device 101 may measure the AGE index 1102 value multiple times and display an average value thereof. The electronic device 101 may display the AGE index 1102 value at various intervals, for example, weekly or monthly.

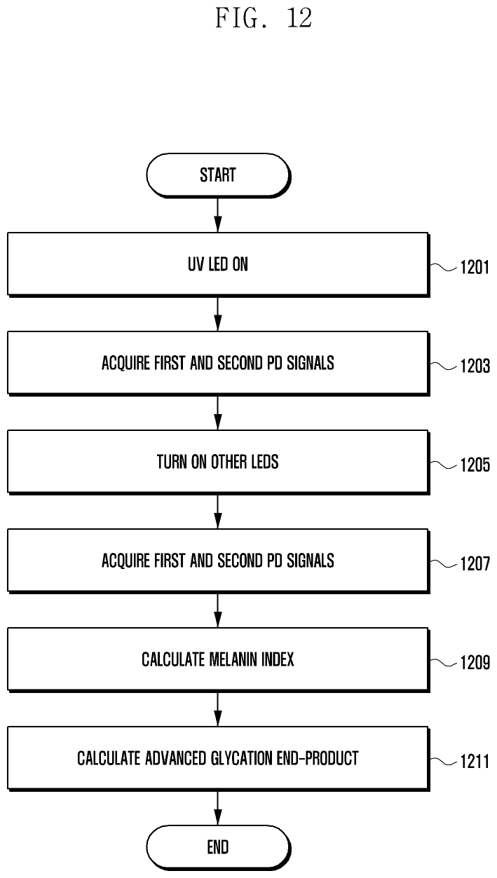

FIG. 12 is a flowchart illustrating a measurement operation of an optical sensor (e.g., the optical sensor 230 in FIGS. 2 to 6) of an electronic device (e.g., the electronic device 101 in FIG. 1 or 2) according to an embodiment of the disclosure.

Referring to FIG. 12, respective operations may be sequentially performed, but are not necessarily sequentially performed. For example, the sequential position of each operation may be changed, or at least two operations may be performed in parallel.

It may be understood that operations 1201 to 1211 are performed by a processor (e.g., the processor 120 in FIG. 1) of the electronic device (e.g., the electronic device 101 in FIG. 1).

The electronic device may perform the measurement operation by using the optical sensor.

The AGE measurement may be performed manually or continuously automatically depending on a user's selection (on-demand). In case of the automatic measurement, the measurement may be performed when a state of little or no movement, such as sleep, lasts for more than a predetermined period of time. During sleep, when movement is minimal or absent, the accuracy of AGE measurement, which requires analysis across multiple wavelengths, may be further improved.

For example, the AGEs may be measured during sleep sessions by conducting measurements for a predetermined period (e.g., 10 seconds) every hour. The AGE values measured during sleep sessions may be used to determine a representative AGE index based on various mathematical operations, such as the average, mode, median, maximum, and/or minimum values.

In operation 1201, the electronic device 101 may operate by turning on a first light-emitting element (e.g., the UV LED) of the optical sensor 230.

In operation 1203, the electronic device 101 may acquire a magnitude of an optical signal emitted according to turning on of the first light-emitting element of the optical sensor 230 through a first light-receiving element and a second light-receiving element.

The electronic device 101 may cause the UV LED of the optical sensor to emit light toward the user's skin to induce AGEs in the skin to react and produce fluorescent light. A magnitude of an optical signal of the UV LED may be measured through the first light-receiving element. The induced fluorescent light of AGE materials may be measured using the second light-receiving element so that a magnitude of a fluorescent signal may be measured.

The electronic device 101 may, for example, sample biometric data at a frequency of about 25 Hz (25 times per second) during the sleep sessions. The electronic device 101 may control LES of UV, R, G, B, and IR to be activated (e.g., turned on to emit light) for measurement. In this case, it is also possible to selectively enable other bands other than the UV band. For example, based on a sampling frequency of about 25 Hz, sampling may be performed at intervals of about 40 ms, during which the UV LED may be activated, followed by the sequential activation of red, green, blue, and IR (infrared) LEDs within about 40 ms. According to a sequential schedule, multiple light-receiving elements may measure the light intensity of activated bands, store the measured values, and use same to calculate the AGE value.

When LEDs are activated, bands with little overlap, rather than bands adjacent to each other, may be activated at the same timing. For example, LEDs having a blue bend and an IR band may be concurrently activated and a PD may measure a signal magnitude through filtering.

The electronic device 101 may sequentially or concurrently activate LEDs (e.g., a red, IR, green, and/or or blue LED) excluding the first light-emitting element in operation 1205 after the operation of the first light-emitting element and may acquire the magnitude of an optical signal through the first light-receiving element and the second light-receiving element in operation 1207. In this case, light emitted from each light-emitting element of the optical sensor may be sensed by a light-receiving element (close PD) disposed adjacent thereto and a light-receiving element (far PD) disposed farther away therefrom.

In case of the green and blue light having relatively short wavelengths, the skin penetration depth is shallow, resulting in higher light amount being detected by nearby light-receiving element (close PD). In contrast, in case of the red or IR light having relatively longer wavelengths, the skin penetration is deep, allowing the signal to travel further after being absorbed, scattered, or reflected within the body and reach a light-receiving element (far PD) disposed at a distance.

In operation 1209, the electronic device 101 may calculate a melanin index by using an optical signal measurement value of another light-emitting element (e.g., the red, IR, green, and/or blue LED).

In operation 1211, the electronic device 101 may estimate an AGE index value based on the fluorescence amount induced using the optical signal measurement value of the first light-emitting element (e.g., the UV LED). Here, the electronic device 101 may correct the fluorescence amount by using the melanin index.

According to an embodiment, a wearable device (e.g., the electronic device 101 in FIG. 1 or 2) may include a housing 200 including a first surface (e.g., the first surface 210 in FIG. 2), a second surface (e.g., the second surface 220 in FIG. 2) opposite the first surface, and a third surface substantially surrounding a space between the first surface and the second surface and configuring a lateral surface of the wearable device, a display received in the housing to be seen through the first surface, a printed circuit board disposed between the display and the second surface, and at least one biometric sensor (e.g., the biometric sensor 230 in FIGS. 2, 3, 4, 5, and 6) disposed on the printed circuit board to face the second surface and including multiple light-emitting elements (e.g., the light-emitting elements 301, 302, 303, 304, 311, 312, 313, 314, 501, and 503 in FIGS. 3, 4, 5, and/or 6) and multiple light-receiving elements (e.g., the light-receiving elements 321, 322, 323, 324, 521, 522, 523, and/or 524 in FIGS. 3, 4, 5, and/or 6), wherein the multiple light-emitting elements comprise a first light-emitting element (e.g., the light-emitting element 301, 302, 303, 304, 501 and/or 503 in FIGS. 3, 4, 5, and/or 6) disposed in a peripheral area of the printed circuit board and configured to emit first light in a first specified band and a second light-emitting element disposed in a center area of the printed circuit board and configured to emit second light in a second specified band at least partially different from the first specified band, the multiple light-receiving elements include a first light-receiving element and a second light-receiving element configured to receive light emitted from the first light-emitting element and the second light-emitting element and reflected from a portion of the user's body, and the first light-emitting element is disposed in the peripheral area to be adjacent to each of the first light-receiving element and the second light-receiving element.

According to an embodiment, the first light-emitting element may be spaced apart a first distance from the first light-receiving element and spaced a second distance apart from the second light-receiving element, the second distance being substantially identical to the first distance.

According to an embodiment, the first light-emitting element may include an ultraviolet (UV) light-emitting element and the second light-receiving element may include a diode configured to filter at least a portion of light in a UV band and receive the filtered light.

According to an embodiment, a partition wall structure configured to optically isolate the first light-emitting element may be further included.

According to an embodiment, the multiple light-emitting elements may further include a third light-emitting element configured to emit light in a substantially identical band to the first light, and the third light-emitting element may be disposed in the peripheral area and positioned to be substantially symmetrical to the first light-emitting element with respect to the center area when viewed from above the second surface.

According to an embodiment, the multiple light-receiving elements may further include a third light-receiving element and a fourth light-receiving element, the third light-receiving element may be positioned to be substantially symmetrical to the first light-receiving element with respect to the center area, and the fourth light-receiving element may be positioned to be substantially symmetrical to the second light-receiving element with respect to the center area.

According to an embodiment, the third light-emitting element may include an ultraviolet (UV) light-emitting element and the fourth light-receiving element may include a diode configured to filter at least a portion of light in a UV band and receive the filtered light.

According to an embodiment, the third light-emitting element may be spaced a third distance apart from the third light-receiving element and spaced a fourth distance apart from the fourth light-receiving element, the fourth distance being substantially identical to the third distance.

According to an embodiment, the multiple light-emitting elements may further include a fourth light-emitting element and a fifth light-emitting element configured to emit light in a third band different from the first band, the fourth light-emitting element and the fifth light-emitting element may be disposed in the peripheral area between the second light-receiving element and the third light-receiving element, and the first light-receiving element may be configured to receive at least a portion of light emitted from the fourth light-emitting element or the fifth light-receiving element and reflected from a portion of the user's body.

According to an embodiment, the first light-emitting element may be configured to be turned on in sequence with at least one of the second light-emitting element, the fourth light-emitting element, or the fifth light-emitting element.

According to an embodiment, at least one of the at least one biometric sensor may be configured to measure an advanced glycation end product (AGE) within the user's skin, at least partially based on the intensity of the reflected light emitted from at least one of the first light-emitting element or the third light-emitting element and acquired through the first light-receiving element or the second light-receiving element.

According to an embodiment, at least one of the at least one biometric sensor may be configured to estimate a melanin index of the user's skin and correct the AGE, at least partially based on the intensity of the reflected light emitted from at least one of the second light-emitting element, the fourth light-emitting element, or the fifth light-emitting element and acquired through the first light-receiving element or the second light-receiving element.

According to an embodiment, the first band may correspond to the UV band, while each of the fourth and fifth bands may correspond to red, blue, green, violet, yellow, or IR (infra-red).

According to an embodiment, at least one of the at least one biometric sensor may be configured to measure an advanced glycation end product (AGE) within the user's skin, at least partially based on the intensity of the reflected light acquired through the first light-receiving element or the second light-receiving element.

According to an embodiment, at least one of the at least one biometric sensor may be configured to estimate a melanin index of the user's skin and correct the AGE, at least partially based on the intensity of the reflected light acquired through the first light-receiving element or the second light-receiving element.

According to an embodiment, the advanced glycation end-product (AGE) may be measured at a specified interval, and an AGE index including one or more values such as the average, maximum, or minimum of the AGE measurement value may be configured to be determined.

According to an embodiment, an electronic device (e.g., the electronic device 101 in FIG. 1 or 2) may include a housing 200 having a transparent cover coming in contact with the body of a user of the electronic device, multiple light-emitting elements (e.g., the light-emitting elements 301, 302, 303, 304, 311, 312, 313, 314, 501, and 503 in FIGS. 3, 4, 5, and/or 6) including a first light-emitting element configured to emit ultraviolet (UV) light through the transparent cover and a second light-emitting element separated from the first light-emitting element by means of a partition wall and configured to emit at least one of visible light or infra-red (IR) light through the transparent cover, multiple light-receiving elements (e.g., the light-receiving elements 321, 322, 323, 324, 521, 522, 523, and/or 524 in FIGS. 3, 4, 5, and/or 6) including a first light-receiving element and a second light-receiving element configured to detect unfiltered UV light and filtered UV light emitted from the multiple light-emitting elements and reflected by a portion of the user's body, respectively, and a processor (e.g., the processor 120 in FIG. 1) configured to acquire first biometric information associated with the user based on the unfiltered UV light and the filtered UV light corresponding to the UV light emitted by the first light-emitting element and acquire second biometric information based on the unfiltered UV light and/or the filtered UV light corresponding to at least one of the visible light or the IR light emitted by the second light-emitting element.

According to an embodiment, the first light-emitting element may be spaced a first distance apart from the first light-receiving element and spaced a second distance apart from the second light-receiving element, and the second distance may be substantially identical to the first distance.

According to an embodiment, the multiple light-emitting elements may further include a third light-emitting element configured to emit UV light through the transparent cover, and the third light-emitting element may be disposed at a position symmetrical to the first light-emitting element.

According to an embodiment, the multiple light-receiving elements may further include a third light-receiving and a fourth light-receiving element configured to detect unfiltered UV light and filtered UV light, emitted from the multiple light-emitting elements and reflected by a portion of the user's body, respectively, the third light-receiving element may be disposed at a position symmetrical to the first light-receiving element, and the fourth light-receiving element may be disposed at a position symmetrical to the second light-receiving element.

According to an embodiment, the third light-emitting element may be spaced a third distance apart from the third light-receiving element and spaced a fourth distance apart from the fourth light-receiving element, and the fourth distance may be substantially identical to the third distance.

According to an embodiment, the processor may be configured to sequentially turn on at least one of the first light-emitting element and the third light-emitting element, and the second light-emitting element.

According to an embodiment, the processor may turn on at least one of the first light-emitting element and the third light-emitting element and acquire the first biometric information at least partially based on an intensity of the light acquired through the first light-receiving element, the second light-receiving element, the third light-receiving element, and the fourth light-receiving element.

According to an embodiment, the processor may turn on the second light-emitting element and acquire the second biometric information at least partially based on an intensity of the light acquired through the first light-receiving element, the second light-receiving element, the third light-receiving element, and the fourth light-receiving element.

According to an embodiment, the first biometric information may include a measurement value of advanced glycation end products (AGEs) within the user's skin, the second biometric information may include the melanin index of the user's skin, and the processor may correct the AGE measurement value based on the melanin index.

According to an embodiment, the processor may be configured to measure the advanced glycation end-product (AGE) at a specified interval, and determine an AGE index including one or more values such as the average, maximum, or minimum of the AGE measurement value.

The electronic device according to various examples may be one of various types of electronic devices. The electronic devices may include, for example, a portable communication device (e.g., a smartphone), a computer device, a portable multimedia device, a portable medical device, a camera, a wearable device, or a home appliance. According to an example of the disclosure, the electronic devices are not limited to those described above.

It should be appreciated that various examples of the disclosure and the terms used therein are not intended to limit the technological features set forth herein to particular examples and include various changes, equivalents, or replacements for a corresponding example. With regard to the description of the drawings, similar reference numerals may be used to refer to similar or related elements. As used herein, each of such phrases as “A or B,” “at least one of A and B,” “at least one of A or B,” “A, B, or C,” “at least one of A, B, and C,” and “at least one of A, B, or C,” may include any one of, or all possible combinations of the items enumerated together in a corresponding one of the phrases. As used herein, such terms as “Ist” and “2nd,” or “first” and “second” may be used to simply distinguish a corresponding component from another, and does not limit the components in other aspect (e.g., importance or order). It is to be understood that if an element (e.g., a first element) is referred to, with or without the term “operatively” or “communicatively”, as “coupled with,” “coupled to,” “connected with,” or “connected to” another element (e.g., a second element), it denotes that the element may be coupled with the other element directly (e.g., wiredly), wirelessly, or via a third element.

As used in connection with various examples of the disclosure, the term “module” may include a unit implemented in hardware, software, or firmware, and may interchangeably be used with other terms, for example, “logic,” “logic block,” “part,” or “circuitry”. A module may be a single integral component, or a minimum unit or part thereof, adapted to perform one or more functions. For example, according to an example, the module may be implemented in a form of an application-specific integrated circuit (ASIC).

Various examples as set forth herein may be implemented as software (e.g., the program 140) including one or more instructions that are stored in a storage medium (e.g., internal memory 136 or external memory 138) that is readable by a machine (e.g., the electronic device 101). For example, a processor (e.g., the processor 120) of the machine (e.g., the electronic device 101) may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a complier or a code executable by an interpreter. The machine-readable storage medium may be provided in the form of a non-transitory storage medium. Wherein, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

According to an example, a method according to various examples of the disclosure may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., PlayStore™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to various examples, each component (e.g., a module or a program) of the above-described components may include a single entity or multiple entities, and some of the multiple entities may be separately disposed in different components. According to various examples, one or more of the above-described components may be omitted, or one or more other components may be added. Alternatively or additionally, a plurality of components (e.g., modules or programs) may be integrated into a single component. In such a case, according to various examples, the integrated component may still perform one or more functions of each of the plurality of components in the same or similar manner as they are performed by a corresponding one of the plurality of components before the integration. According to various examples, operations performed by the module, the program, or another component may be carried out sequentially, in parallel, repeatedly, or heuristically, or one or more of the operations may be executed in a different order or omitted, or one or more other operations may be added.

While the disclosure has been shown and described with reference to various embodiments thereof, it will be understood by those skilled in the art that various changes in form and details may be made therein without departing from the spirit and scope of the disclosure as defined by the appended claims and their equivalents.

What is claimed is:

1. A wearable device comprising:
a housing comprising:
a first surface,
a second surface opposite the first surface, and
a third surface substantially surrounding a space between the first surface and the second surface and forming a lateral surface of the wearable device;
a display received in the housing to be seen through the first surface;
a printed circuit board disposed between the display and the second surface; and
at least one biometric sensor disposed on the printed circuit board to face the second surface and comprising multiple light-emitting elements and multiple light-receiving elements,
wherein the multiple light-emitting elements comprise:
a first light-emitting element disposed in a peripheral area of the printed circuit board and configured to emit first light in a first specified band, and
a second light-emitting element disposed in a center area of the printed circuit board and configured to emit second light in a second specified band at least partially different from the first specified band, and
wherein the multiple light-receiving elements comprise:
a first light-receiving element, and
a second light-receiving element configured to receive light emitted from the first light-emitting element and the second light-emitting element and reflected from a portion of a user's body, the first light-emitting element being disposed in the peripheral area to be adjacent to each of the first light-receiving element and the second light-receiving element.

2. The wearable device of claim 1, wherein the first light-emitting element is spaced a first distance apart from the first light-receiving element and spaced a second distance apart from the second light-receiving element, the second distance being substantially identical to the first distance.

3. The wearable device of claim 1, wherein the first light-emitting element is configured to emit ultraviolet (UV) light and the second light-receiving element is configured to filter at least a portion of light in a UV band and receive the filtered light.

4. The wearable device of claim 1, further comprising:
a partition wall structure configured to optically isolate the first light-emitting element.

5. The wearable device of claim 1, wherein the multiple light-emitting elements further comprise a third light-emitting element configured to emit light in a substantially identical band to the first light, the third light-emitting element being disposed in the peripheral area and positioned to be substantially symmetrical to the first light-emitting element with respect to the center area when viewed from above the second surface.

6. The wearable device of claim 5, wherein the multiple light-receiving elements further comprise a third light-receiving element and a fourth light-receiving element, the third light-receiving element being positioned to be substantially symmetrical to the first light-receiving element with respect to the center area, and the fourth light-receiving element being positioned to be substantially symmetrical to the second light-receiving element with respect to the center area.

7. The wearable device of claim 6,
wherein the third light-emitting element comprises an ultraviolet (UV) light-emitting element, and
wherein the fourth light-receiving element comprises a diode configured to filter at least a portion of light in a UV band and receive the filtered light.

8. The wearable device of claim 6, wherein the third light-emitting element is spaced a third distance apart from the third light-receiving element and spaced a fourth distance apart from the fourth light-receiving element, the fourth distance being substantially identical to the third distance.

9. The wearable device of claim 6, wherein the multiple light-emitting elements further comprise a fourth light-emitting element and a fifth light-emitting element configured to emit light in a third band different from the first band, the fourth light-emitting element and the fifth light-emitting element being disposed in the peripheral area between the second light-receiving element and the third light-receiving element, and the first light-receiving element being configured to receive at least a portion of light emitted from the fourth light-emitting element or the fifth light-receiving element and reflected from a portion of the user's body.

10. The wearable device of claim 9,
wherein the first light-emitting element is configured to be turned on in sequence with at least one of the second light-emitting element, the fourth light-emitting element, or the fifth light-emitting element,
wherein at least one of the at least one biometric sensor is configured to measure an advanced glycation end product (AGE) within the user's skin, at least partially based on an intensity of the reflected light, emitted from at least one of the first light-emitting element or the third light-emitting element and acquired through the first light-receiving element or the second light-receiving element, and
wherein at least one of the at least one biometric sensor is configured to estimate a melanin index of the user's skin and correct the AGE, at least partially based on an intensity of the reflected light, emitted from at least one of the second light-emitting element, the fourth light-emitting element, or the fifth light-emitting element and acquired through the first light-receiving element or the second light-receiving element.

11. An electronic device comprising:
a housing having a transparent cover coming in contact with a body of a user of the electronic device;
multiple light-emitting elements comprising:
a first light-emitting element configured to emit ultraviolet (UV) light through the transparent cover, and
a second light-emitting element separated from the first light-emitting element by means of a partition wall and configured to emit at least one of visible light or infra-red (IR) light through the transparent cover;
multiple light-receiving elements comprising:
a first light-receiving element, and
a second light-receiving element configured to detect unfiltered UV light and filtered UV light, emitted from the multiple light-emitting elements and reflected by a portion of the body of the user, respectively;
memory storing one or more computer programs; and
one or processors communicatively coupled to the memory,
wherein the one or more computer programs include computer-executable instructions that, when executed by the one or more processors individually or collectively, cause the electronic device to:
acquire first biometric information associated with the user, based on the unfiltered UV light and the filtered UV light corresponding to the UV light emitted by the first light-emitting element, and
acquire second biometric information, based on the unfiltered UV light and/or the filtered UV light corresponding to at least one of the visible light or the IR light emitted by the second light-emitting element.

12. The electronic device of claim 11, wherein the first light-emitting element is spaced a first distance apart from the first light-receiving element and spaced a second distance apart from the second light-receiving element, the second distance being substantially identical to the first distance.

13. The electronic device of claim 11, wherein the multiple light-emitting elements further comprise a third light-emitting element configured to emit UV light through the transparent cover, the third light-emitting element being disposed at a position symmetrical to the first light-emitting element.

14. The electronic device of claim 13, wherein the multiple light-receiving elements further comprise a third light-receiving and a fourth light-receiving element configured to detect unfiltered UV light and filtered UV light, emitted from the multiple light-emitting elements and reflected by a portion of the body of the user, respectively, the third light-receiving element being disposed at a position symmetrical to the first light-receiving element, and the fourth light-receiving element being disposed at a position symmetrical to the second light-receiving element.

15. The electronic device of claim 14, wherein the third light-emitting element is spaced a third distance apart from the third light-receiving element and spaced a fourth distance apart from the fourth light-receiving element, the fourth distance being substantially identical to the third distance.

16. The electronic device of claim 11, wherein the transparent cover forms part of a rear surface of the housing.

17. The electronic device of claim 11, wherein the multiple light-emitting elements are arranged in a peripheral area of an optical sensor of the electronic device.

18. One or more non-transitory computer-readable storage media storing one or more computer programs including computer-executable instructions that, when executed by one or more processors of an electronic device individually or collectively, cause the electronic device to perform operations, the operations comprising:
acquiring first biometric information associated with a user, based on unfiltered UV light and filtered UV light corresponding to UV light emitted by a first light-emitting element of the electronic device, and
acquiring second biometric information, based on the unfiltered UV light and/or the filtered UV light corresponding to at least one of visible light or infrared light emitted by a second light-emitting element of the electronic device.
